(19) United States
(12) Patent Application Publication
(10) Pub. No.: US 2026/0253706 A1
(43) Pub. Date: Aug. 27, 2026

(54) System and Method for AI-Powered Fitness Tracking Platform

(71) Applicant: Cameron Immesoete, Orinda, CA (US)

(72) Inventor: Cameron Immesoete, Orinda, CA (US)

(21) Appl. No.: 19/060,734

(22) Filed: Feb. 23, 2025

(51) Int. Cl. G16H 20/60; G06F 16/9535; G06V 10/764; G16H 20/30; H04L 51/02

(52) U.S. Cl. CPC G16H 20/60; G06F 16/9535; G06V 10/764; G16H 20/30; H04L 51/02

(57) ABSTRACT

Implementations described herein relate to methods, systems, and computer programs for an AI-powered fitness tracking platform that automatically processes dietary information from food images, logs user metrics (e.g., weight, body measurements, etc.), and employs generative AI to provide personalized nutrition and fitness recommendations. By capturing a photo of a meal, the system identifies and logs calories, macronutrients, and micronutrients. Users can also enter data manually, search an existing nutritional database, and interact with a generative AI chatbot to modify or enhance data entries.

### TECHNICAL FIELD

Embodiments relate generally to health and fitness technology, artificial intelligence, and dynamic user-data management, and more particularly, to methods, systems, and computer-readable media for capturing images of meals to automatically analyze and record nutritional data, while providing an AI-driven conversational interface for personalized recommendations and logging adjustments.

### BACKGROUND

Conventional fitness and diet-tracking applications often require extensive manual data entry for meal logging. While databases can streamline nutritional lookup, users still spend considerable effort entering details such as servings, ingredient breakdowns, or tracking various micronutrients. Moreover, obtaining personalized recommendations typically requires either manual research or reliance on static meal plans. Recent advances in computer vision and large language models (LLMs) enable automated meal recognition, nutrient estimation, and responsive, conversational user interaction.

### SUMMARY

According to an aspect, a computer-implemented method for AI-powered fitness tracking and personalized guidance is provided. The method includes:
Capturing an image of a meal via a user device.
Analyzing the image using Al-based computer vision to determine calories, macronutrients, and micronutrients.
Storing the extracted data in a user-specific log that also tracks metrics such as weight, height, body measurements, and historical trends.
Employing a generative Al chatbot to provide personalized dietary or fitness suggestions, dynamically update entries, and respond to user inquiries.
Allowing manual data entry and a searchable nutritional database for cases where the system cannot identify certain foods or for user preferences.

This approach automates meal logging, saves time for users, and offers tailored health insights based on user-specific goals and preferences.

### BRIEF DESCRIPTION OF THE DRAWINGS

FIG. 1 is a system architecture diagram illustrating how user devices, an Al nutrition analysis module, a generative Al chatbot, and a user database interact. The user device captures meal images, and the system analyzes the images, updates logs, and provides personalized recommendations.

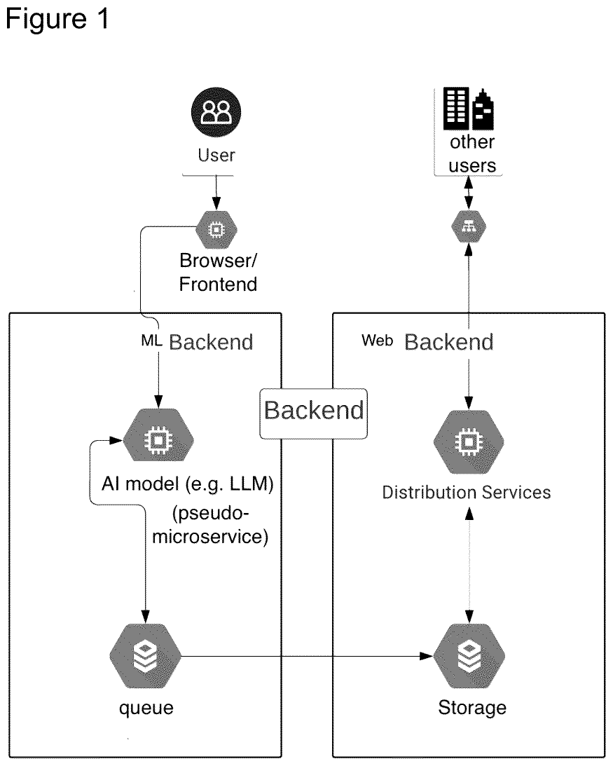

1. A computer-implemented method for AI-powered fitness tracking, the method comprising:
a. receiving, from a user device, at least one image of a meal;
analyzing the at least one image using an artificial intelligence model to estimate nutritional information comprising calories, macronutrients, and micronutrients;
b. logging the estimated nutritional information in association with user-specific data including weight, height, age, and body measurements;
c. generating personalized dietary or fitness recommendations through a generative AI chatbot based on the nutritional information and the user-specific data; and
d. enabling manual modification or entry of nutritional information, including searching an existing nutrition database and conversing with the generative AI chatbot to adjust logged data.

2. The method of claim 1, further comprising storing historical meal data, body measurements, and progress metrics in a repository accessible by the generative AI chatbot for adaptive recommendation generation.

3. The method of claim 1, wherein the analyzing step includes identifying multiple food items in a single image and allocating estimated nutritional content for each identified item.

4. The method of claim 1, wherein the generative AI chatbot utilizes user preferences, dietary restrictions, and real-time nutrient gaps to suggest specific food items to optimize daily nutrient intake.

5. The method of claim 1, further comprising enabling a user to override system-generated nutritional estimates by submitting textual or voice-based clarifications to the generative AI chatbot, which updates the corresponding meal log entry.

6. The method of claim 1, wherein the user device comprises a mobile application or web-based interface through which the user initiates image capture, reviews logged data, and communicates with the chatbot.
